(19) United States
(12) Patent Application Publication
(10) Pub. No.: US 2022/0265415 A1
(43) Pub. Date: Aug. 25, 2022

(54) TISSUE REGENERATION PLATFORM INCLUDING HYALURONIC ACID-CATECHOL COMPOUND AND PREPARATION METHOD THEREOF

(71) Applicants: SEOUL NATIONAL UNIVERSITY R&DB FOUNDATION, Seoul (KR); WONKWANG UNIVERSITY CENTER FOR INDUSTRY-ACADEMY COOPERATION, Iksan-si Jeollabuk-do (KR)

(72) Inventors: Kyung Pyo PARK, Seoul (KR); Sang Woo LEE, Seoul (KR); Ji Hyun RYU, Daejeon (KR)

(73) Assignees: SEOUL NATIONAL UNIVERSITY R&DB FOUNDATION, Seoul (KR); WONKWANG UNIVERSITY CENTER FOR INDUSTRY-ACADEMY COOPERATION, Iksan-si Jeollabuk-do (KR)

(21) Appl. No.: 17/639,463

(22) PCT Filed: Jun. 17, 2020
(86) PCT No.: PCT/KR2020/007820

§ 371 Date: Mar. 1, 2022

(30) Foreign Application Priority Data
Sep. 2, 2019 (KR) 10-2019-0108175

(51) Int. Cl. A61F 2/02; A61L 27/20; A61L 27/38; A61L 27/56; A61L 27/54; A61L 27/52

(52) U.S. Cl. CPC A61F 2/022; A61L 27/20; A61L 27/3895; A61L 27/56; A61L 27/54; A61L 27/52

(57) ABSTRACT

The tissue regeneration platform of one embodiment may exhibit excellent biocompatibility and tissue culture property on various supports, comprising a support and a coating layer disposed on the support, and comprising a hyaluronic acid-catechol compound.

### BACKGROUND OF THE INVENTION

### 1. Field of the Invention

The present invention relates to a tissue regeneration platform comprising a hyaluronic acid-catechol compound and a method for fabricating the same. More particularly, it relates to a tissue regeneration platform having improved tissue culture property, comprising a coating layer comprising a hyaluronic acid-catechol compound, and a method for fabricating the same.

### 2. Description of the Related Art

Tissue regeneration is one of the treatments used to restore the function of damaged or degenerated tissues. For tissue regeneration, it is necessary to develop a platform for stably culturing and providing tissue cells.

Recently, in order to fundamentally treat the deterioration of salivary gland function, research on culturing and transplanting salivary glands in hyaluronic acid, hydrogel or the like is being actively conducted. However, since hyaluronic acid is very expensive, a high concentration of hyaluronic acid is required to form a hydrogel, which is not economical. In addition, hyaluronic acid hydrogel has weak strength and weak tissue adsorption, so it is difficult to maintain its shape for a long time in a desired place. On the other hand, materials such as polycaprolactone (PCL) are FDA-approved products and have the advantage of being able to 3D print in the same shape as the damaged area due to their high biocompatibility, and its mechanical strength is high and resistance to external impact is high. However, since the surface of PCL is hard and hydrophobic, it is an environment in which salivary gland cells cannot grow properly.

On the other hand, in the case of agarose gel, alginate gel, etc., it is biocompatible, its strength can be freely adjusted, and the price is low, but there is a problem that the salivary gland does not grow well as the mechanical strength increases.

Therefore, there is a demand for the development of a platform for tissue culture that can utilize the advantages of hyaluronic acid while using a support with secured mechanical strength.

An object of the present invention is to provide a tissue regeneration platform having good mechanical strength and excellent tissue adsorption and high biocompatibility.

An object of the present invention is to provide a tissue regeneration platform capable of culturing stable salivary glands and a method for fabricating the same.

Another object of the present invention is to provide a tissue regeneration platform for culturing an embryonic salivary gland in vitro and applying it to a patient, and a method for fabricating the same, in order to provide a fundamental treatment for a patient with salivary gland dysfunction.

### SUMMARY OF THE INVENTION

One embodiment of the present invention for solving the above technical problem provides a tissue regeneration platform comprising: a support; and a coating layer disposed on the support and comprising a hyaluronic acid-catechol compound.

One embodiment may comprise tissue cells for culture provided on the coating layer.

A salivary gland can be cultured on the coating layer.

The support may be polycarbonate, agarose gel, alginate hydrogel, or polycaprolactone.

The support may be a polymer membrane, a polymer gel, or a 3D polymer scaffold.

The hyaluronic acid-catechol compound may be represented by the following Chemical Formula 1, in which X and Y are each independently an integer of 1 or more and 300 or less.

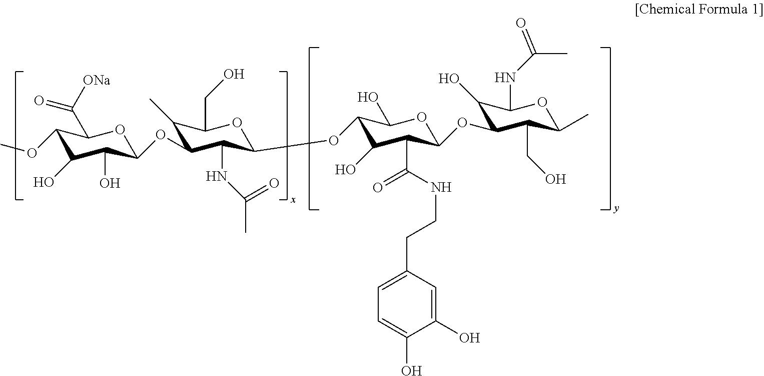

Another embodiment provides a method of fabricating a tissue regeneration platform, the method comprising preparing a mixed solution comprising hyaluronic acid-catechol; and providing the mixed solution on a support to form a coating layer.

One embodiment may comprise culturing a salivary gland on the coating layer.

The support is a polymer membrane, a polymer gel, or a 3D polymer scaffold, and forming the coating layer may comprise immersing the support in the mixed solution.

### EFFECT OF THE INVENTION

The tissue regeneration platform of one embodiment may exhibit excellent tissue culture property and high biocompatibility in various supports by comprising a coating layer comprising hyaluronic acid-catechol on the support.

In addition, the method of fabricating a tissue regeneration platform of one embodiment may provide a tissue regeneration platform that is compatible with culturing of salivary glands by comprising the step of forming a coating layer comprising hyaluronic acid-catechol on various supports.

### BRIEF DESCRIPTION OF THE DRAWINGS

FIG. 1 shows the NMR measurement results of hyaluronic acid and the synthesized hyaluronic acid-catechol compound.

FIG. 2 is a graph showing absorbance in the ultraviolet-visible region.

FIG. 3 is a diagram schematically illustrating the step of forming a coating layer comprising a hyaluronic acid-catechol compound.

FIG. 4 is an image showing the surface state after Alcian blue staining.

FIG. 5 shows a comparison of the element distribution of the surface analyzed by EDS.

FIG. 6 shows the results of the water contact angle test.

FIG. 7 shows a comparison of the culture state of embryonic salivary glands in Comparative Examples and Examples.

### DETAILED DESCRIPTION OF THE PREFERRED EMBODIMENTS

Objects and advantages of the present invention, and technical configurations for achieving them, will become apparent with reference to the embodiments described in detail below in conjunction with the accompanying drawings. In the description of the present invention, when it is determined that a specific description of a known function or configuration may unnecessarily obscure the gist of the present invention, the detailed description thereof will be omitted. And the following terms are defined terms in consideration of the donation in the present invention, which may vary according to the intent or practice of the user etc.

However, it should be understood that the present invention is not limited to the embodiments disclosed below, but may be embodied in a variety of different forms, and includes all modifications, equivalents and substitutes included in the spirit and scope of the present invention. It is to be understood that the embodiments are provided so as to complete the disclosure of the present invention and to fully illuminate the scope of the invention to one of ordinary skill in the art, and that the invention is only defined by the terms of the claims, so that the definitions should be made on the basis of the disclosure throughout the specification.

In addition, similar reference numerals are used for similar components while describing each drawing. In the accompanying drawings, the dimensions of the structures are enlarged than the actual size for clarity of the present invention. In this specification, the singular expression includes the plural expression unless the context clearly dictates otherwise.

In addition, throughout the specification, when a portion is said to “comprise” or “include” a component, this means that the component may further comprise other components but not other components unless specifically stated to the contrary. Further, when a portion of a layer, film, region, plate, etc. is described as being “on” another portion, it includes not only the case where the other portion is “directly on” but also the case when there is another portion in the middle thereof

Hereinafter, a tissue regeneration platform and a method for fabricating the same according to an embodiment of the present invention will be described.

A tissue regeneration platform according to an embodiment of the present invention may comprise a support and a coating layer disposed on the support and comprising a hyaluronic acid-catechol compound. The coating layer comprising the hyaluronic acid-catechol compound may cover the support and constitute an outer surface of the support.

The hyaluronic acid-catechol compound may be a hyaluronic acid polymer comprising a catechol group. The hyaluronic acid-catechol compound is a hyaluronic acid compound having adhesive properties. The hyaluronic acid-catechol compound may be coated on the surface of various materials, and the coating layer comprising the hyaluronic acid-catechol compound may exhibit adhesion suitable for tissue culture and compatibility with tissue cells for culture.

Tissue cells for tissue culture may be provided on the coating layer comprising a hyaluronic acid-catechol compound. Meanwhile, the tissue regeneration platform of one embodiment may be for culturing salivary glands. Thus, salivary glands can be cultured on the coating layer comprising the hyaluronic acid-catechol compound.

On the other hand, the “salivary gland” is an organ that produces and secretes saliva, and can be classified major salivary gland such as parotid gland, submaxillary gland, and sublingual gland, and a minor salivary gland or the like distributed at various sites of the aqueous membrane of the oral cavity, such as a mucous gland present in the oral membrane of cavity.

In an embodiment of the tissue regeneration platform, the support may be a polymer support. For example, polycarbonate, agarose gel, alginate hydrogel, polycaprolactone, etc. may be used as the support, but the embodiment is not limited thereto.

In addition, in the tissue regeneration platform of one embodiment, the support may be a polymer membrane, a polymer gel, or a 3D polymer scaffold. However, the embodiment is not limited thereto, and a polymer scaffold provided in various forms may be used as a support for the tissue regeneration platform.

The coating layer provided on the support may be formed from a hyaluronic acid-catechol compound represented by the following Chemical Formula (1).

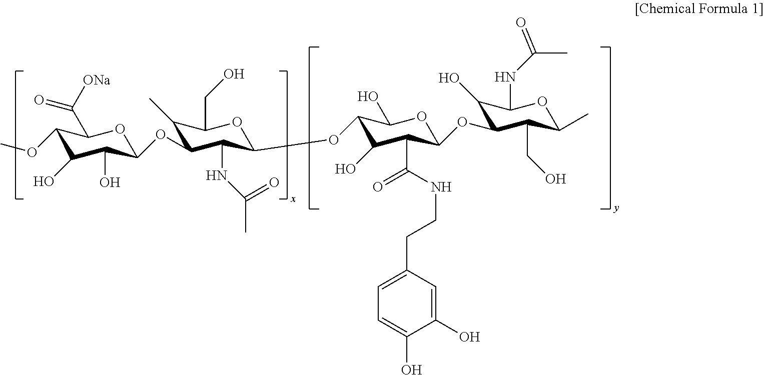

wherein, each of x and y may be an integer of 1 or more and 300 or less.

The coating layer formed from the hyaluronic acid-catechol compound may exhibit hydrophilicity. Therefore, tissue cells and the like can exhibit high adhesion to the coating layer comprising the hyaluronic acid-catechol compound.

The tissue regeneration platform of one embodiment may be for culturing embryonic salivary glands. The hyaluronic acid-catechol used in the present invention is based on hyaluronic acid existing in the environment surrounding the salivary gland during the development of the salivary gland, and can stably coat various surfaces regardless of the type and strength of the surface, and thus stably produce a large amount of salivary gland.

In particular, the salivary glands differ greatly in growth and differentiation depending on the living/physical properties of the environment, which limits the choice of support material for culture. For example, salivary glands do not grow well on hard surfaces and have limitations in that they cannot grow on surfaces such as polycaprolactone or polyvinyl alcohol. However, the coating of hyaluronic acid-catechol on materials in which the salivary glands did not grow well, such as the tissue regeneration platform presented in the present invention, can eliminate the negative effect of the surface properties of such support materials. Therefore, when the tissue regeneration platform of the present invention is used, even a biomaterial which has been previously determined not to be suitable for salty gland culture can be changed to a material which is compatible with the culture of salty land while maintaining the physical properties of the material without simple coating treatment. Therefore, the tissue regeneration platform of the present invention can be said to have excellent versatility.

The tissue regeneration platform of one embodiment can be used as a therapeutic agent capable of regenerating the salivary gland in patients with salivary gland dysfunction that may occur after radiation therapy.

The tissue regeneration platform of one embodiment prepared by culturing embryonic salivary gland can be used for the purpose of ameliorating or treating symptoms that occur when the functional deterioration of the salivary gland occurs. Xerostomia, which is a representative symptom of decreased salivary gland function, causes discomfort when eating or eating food, and also causes oral-related diseases such as bacterial infections, bad breath, etc. Although various reports have been made on the cause of xerostomia, it is often caused by long-term drug treatment such as radiation after anticancer treatment or anticancer agent. To date, there is no fundamental treatment method for treating xerostomia, and a lubricant or saliva component is used as an auxiliary, but there is a limit in increasing salivary secretion. In addition, while radioprotectors have been used to prevent xerostomia that occurs after radiation treatment, various side effects such as hypotension, parasympathetic nerve stimulation have occurred, and therapeutic effects have also been insignificant or limited. Therefore, the method of transplanting an externally cultured artificial salivary gland is suggested as the most fundamental solution.

Accordingly, the tissue regeneration platform of one embodiment prepared by culturing embryonic salivary glands can be used as an artificial salivary gland as a therapeutic agent for alleviating xerostomia.

The method of fabricating a tissue regeneration platform of one embodiment may comprise preparing a mixed solution comprising hyaluronic acid-catechol, and providing the prepared mixed solution on a support to form a coating layer. The method of fabricating a tissue regeneration platform of one embodiment may comprise culturing a salivary gland on the coating layer. For example, by culturing embryonic salivary glands on a coating layer, a tissue regeneration platform for therapeutic use can be fabricated to improve salivary gland dysfunction.

In one embodiment, the mixed solution may be prepared by mixing the hyaluronic acid-catechol compound with DMEM (Dulbeco's Modified Eagle's Media/F12 1:1 1X without phenol red).

A coating layer comprising a hyaluronic acid-catechol compound may be formed by coating the mixed solution on a support. In this case, the support used may be a polymer membrane, a polymer gel, or a 3D polymer scaffold.

Meanwhile, the step of providing the mixed solution on the support to form the coating layer may comprise immersing the support in the mixed solution. That is, the support may be immersed in the mixed solution to form a coating layer covering the support.

For example, the support may be a polycarbonate membrane (PC membrane), an agarose gel, a three-dimensional polycaprolactone scaffold (3D PCL scaffold), etc. A support such as a three-dimensional polycaprolactone scaffold (3D PCL scaffold) may be prepared by a three-dimensional printing method, etc.

The support of the three-dimensional polymer scaffold may be prepared in the form of a nanostructure having porosity. The support of the three-dimensional polymer scaffold may be provided by woven stems in the form of fibers in a matrix form. Hyaluronic acid-catechol may form a coating layer around the fibrous stems.

One embodiment of the present invention may provide a tissue regeneration platform using various types of scaffolds comprising a coating layer comprising hyaluronic acid-catechol. In addition, it is possible to provide a tissue regeneration platform with improved tissue culture characteristics by exhibiting excellent biocompatibility under the influence of hyaluronic acid-catechol.

Hereinafter, with reference to Examples and Comparative Examples, the tissue regeneration platform of an embodiment manufactured using the tissue regeneration platform according to an embodiment of the present invention and the method of fabricating a tissue regeneration platform of the embodiment will be described in detail. The following examples are merely illustrative for facilitating the understanding of the present invention, and the scope of the invention is not limited thereto.

### EXAMPLES

### 1. Synthesis of Hyaluronic Acid-Catechol Compounds

The hyaluronic acid-catechol compound used in one embodiment may be synthesized by the following Reaction Scheme 1.

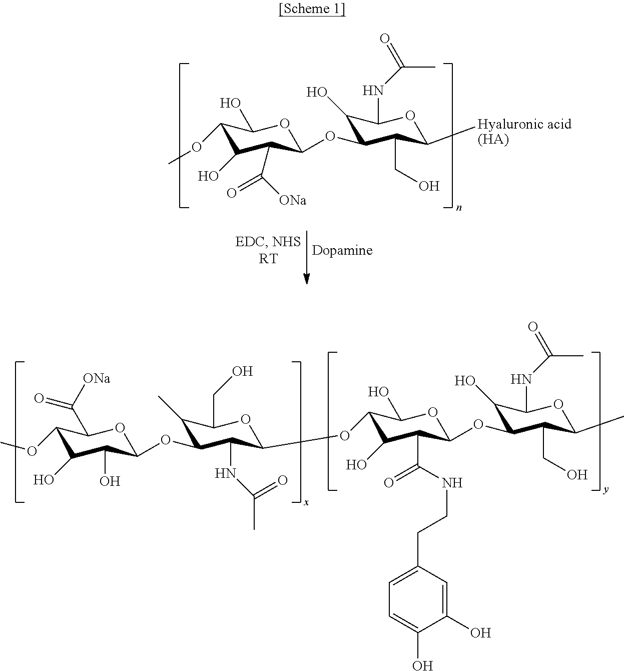

A solution of 1 g of sodium hyaluronate in 100 mL of pH 5.0 MES (2-(N-morpholino)ethanesulfonic acid) buffer was prepared, and 474 mg of 1-ethyl-3-(3-dimethylaminopropyl) (1-Ethyl-3-(3-dimethylaminopropyl)carbodiimide; EDC) and 285 mg of n-hydroxysuccinimide (NHS) were added and maintained for 30 minutes. 388 mg of dopamine hydrochloride was added to the mixed solution, and the pH was maintained at 4.5 to 5.5 for 12 hours. Then, after 3 days of purification using membrane dialysis, freeze-drying was performed to obtain a hyaluronic acid-catechol compound.

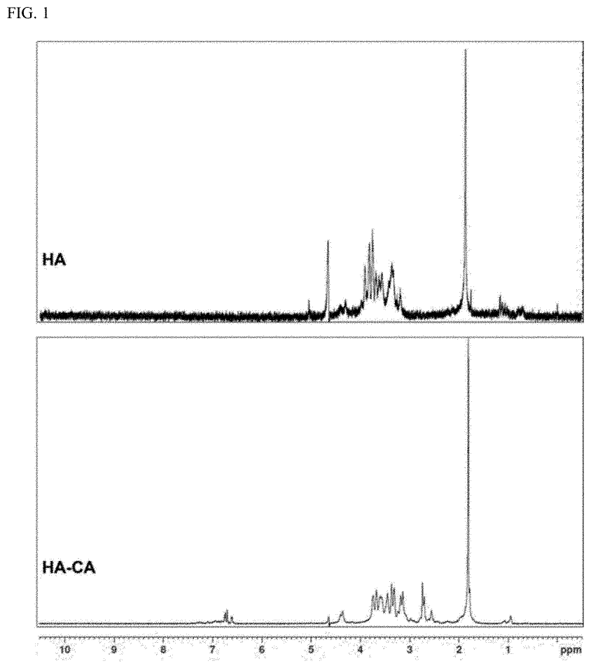

FIG. 1 shows a comparison of NMR measurement results of hyaluronic acid and the synthesized compound. It was confirmed that the synthesized compound from the NMR measurement result of FIG. 1 was a hyaluronic acid-catechol compound.

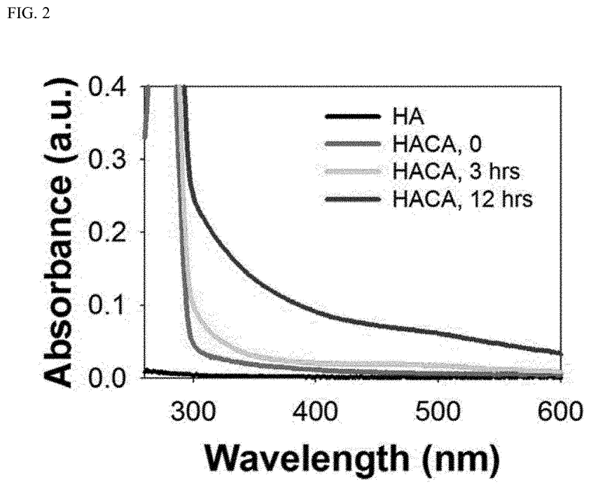

In addition, FIG. 2 is a graph showing the change in absorbance in the ultraviolet-visible region with time. In FIG. 2, “HA” indicates the absorption spectrum of hyaluronic acid, and “HACA” indicates the change in the absorption spectrum according to the reaction time according to Scheme 1 above. Referring to FIG. 2, it can be confirmed that the amount of synthesized hyaluronic acid-catechol is increased from the increase in absorbance in the ultraviolet-visible region of 300 nm to 600 nm as time passes.

### 2. Fabrication of Tissue Regeneration Platform

A tissue regeneration platform was prepared by coating, on the support, the mixed solution comprising the hyaluronic acid-catechol compound prepared by the above-described synthesis method.

The mixed solution can be prepared by mixing the hyaluronic acid-catechol compound with high temperature sterilized DMEM (Dulbeco's Modified Eagle's Media/F12 1:1 1X without phenol red) at a concentration of 2 to 5 mg/mL. After the mixed solution was stirred at room temperature for 12 hours, the support was immersed in the stirred mixed solution to form a coating layer on the support.

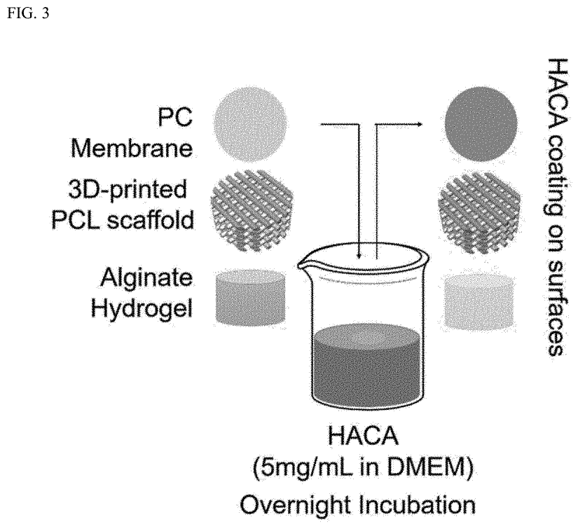

FIG. 3 is a diagram schematically illustrating the step of forming, on a support, a coating layer comprising a hyaluronic acid-catechol compound. Referring to FIG. 3, the coating layer may be provided by immersing the support in a mixed solution comprising hyaluronic acid-catechol. For example, the support may be a polycarbonate (PC) membrane, a polycaprolactone scaffold formed by 3D printing, or an alginate hydrogel. However, the embodiment is not limited thereto, and various types of polymer supports may be used. The polymer support may be provided in various forms such as a porous membrane, a porous three-dimensional structure, or a block shape.

The surface of the support immersed in the mixed solution was then washed 3-4 times using sterilized tertiary distilled water, and then dried at room temperature to prepare a tissue regeneration platform.

### 3. Confirmation of Surface Properties of Tissue Regeneration Platform

To confirm the physical properties of the coating layer comprising the hyaluronic acid-catechol compound, the surface state after Alcian blue staining was observed, and scanning electron microscopy (SEM) and Energy Dispersive Spectroscopy (EDS), and water contact angle tests were conducted to confirm whether a coating layer comprising hyaluronic acid-catechol compounds was formed.

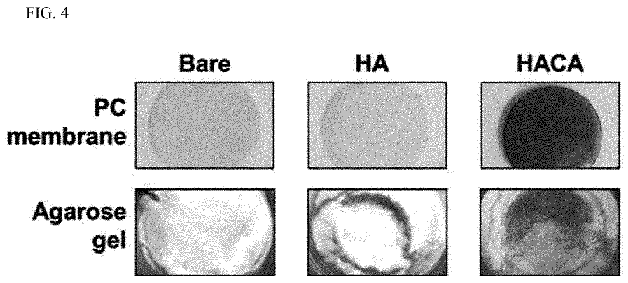

FIG. 4 is an image showing the surface state after Alcian blue staining. As a support, a polycarbonate membrane and an agarose gel were used. In the image shown in FIG. 4, “Bare” is a case in which a coating layer is not provided on a support, “HA” is a case in which hyaluronic acid is coated on a support, and “HACA” is a case in which hyaluronic acid-catechol is coated on a support. In case of using both polycarbonate membrane and agarose gel, it can be seen that the adhesion to the support is high when hyaluronic acid-catechol is coated on the support from the fact that a lot of blue-dyed parts appear in “HACA”.

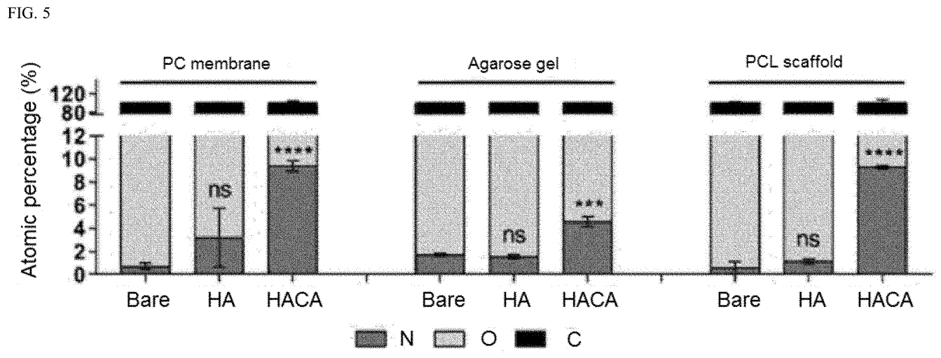

FIG. 5 shows a comparison of the element distribution of the surface layer analyzed by EDS. In FIG. 5, N, O, and C each represent elemental distribution ratios of nitrogen atoms, oxygen atoms, and carbon atoms on the surface. On the other hand, when hyaluronic acid is distributed on the surface, the distribution ratio of nitrogen atoms (N) contained in the hyaluronic acid is high. The results of FIG. 5 compare the coating degree of hyaluronic acid in each of the polycarbonate membrane, agarose gel, and polycaprolactone scaffold. In FIG. 5, “Bare” is a case in which a coating layer is not provided on a support, “HA” is a case in which hyaluronic acid is coated on a support, and “HACA” is a case in which hyaluronic acid-catechol is coated on a support.

Referring to the results of FIG. 5, when three different supports were used, a high nitrogen atom distribution ratio was exhibited in “HACA”. From this, it can be confirmed that when hyaluronic acid-catechol is coated on a support, adhesion to the support is excellent.

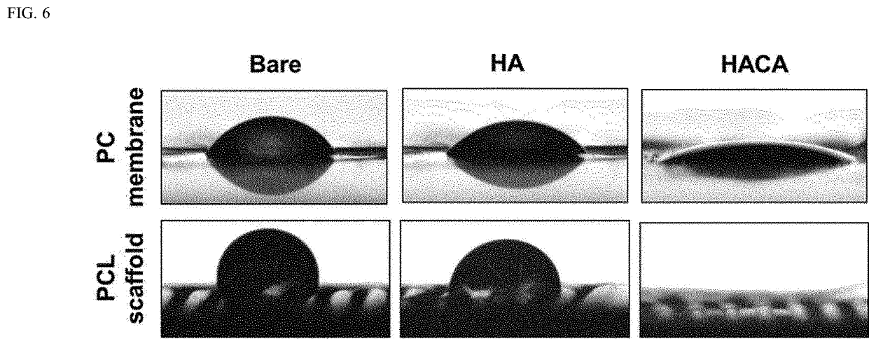

FIG. 6 shows the results of the water contact angle test. In the water contact angle test, the larger the contact angle, the more hydrophobic, the smaller the contact angle, the more hydrophilic. In FIG. 6, “Bare” is a case in which a coating layer is not provided on a support, “HA” is a case in which hyaluronic acid is coated on a support, and “HACA” is a case in which hyaluronic acid-catechol is coated on a support.

The result of FIG. 6 shows the water contact angle in each of the polycarbonate membrane and the polycaprolactone scaffold, and it can be confirmed that in both cases, a small contact angle was shown in “HACA”. That is, when hyaluronic acid-catechol is used, it can be confirmed that the surface can maintain hydrophilicity by increasing the bonding strength of hyaluronic acid on the support.

### 4. Culture Characteristics of Salivary Gland

Comparative examples of embryonic salivary glands growing on the surface of existing materials (indicated by “Bare”) and examples of embryonic salivary glands growing on a surface coated with hyaluronic acid-catechol (indicated as “HACA”) were compared and observed.

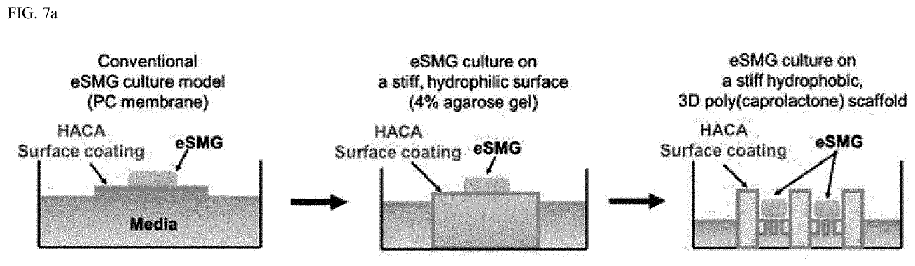

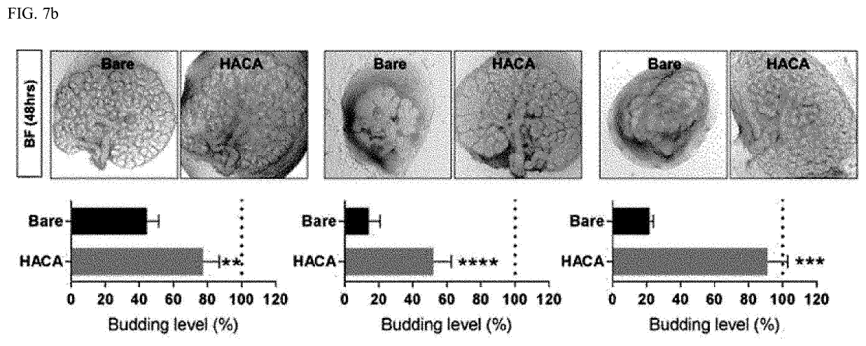

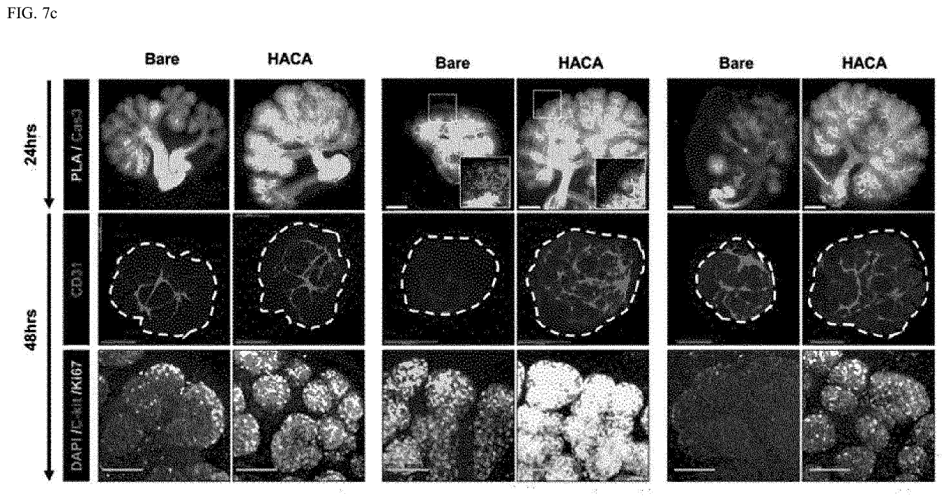

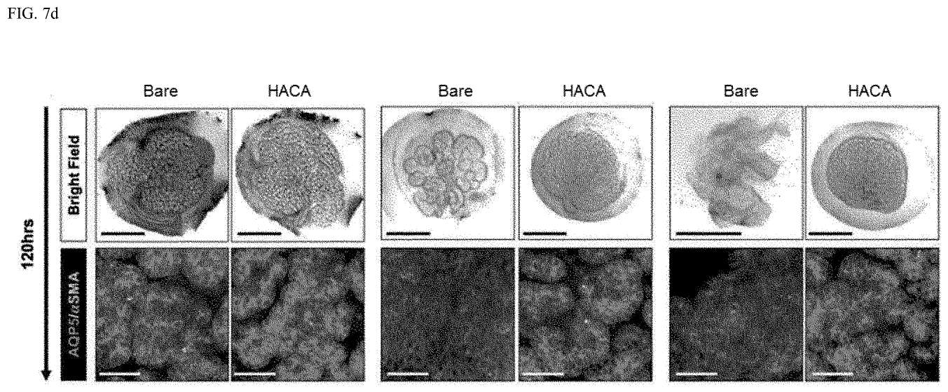

In “a” of FIG. 7, the shape of the tissue regeneration platform according to the type of support for culturing the embryonic salivary gland is schematically shown. First, the effect of hyaluronic acid coated on the surface was evaluated by coating a hyaluronic acid-catechol compound on the air-media interface using a polycarbonate membrane, which is the most common method for culturing embryonic salivary glands. Next, a hyaluronic acid-catechol compound was coated on the hard agarose hydrogel and polycaprolactone scaffold surface to evaluate whether the coated hyaluronic acid affects the growth of embryonic salivary glands.

Referring to FIG. 7, the embryonic salivary gland grown on the surface coated with hyaluronic acid-catechol showed a higher number of epithelial buds, the number of blood vessels, and the number of stem cells and higher division activity (b, c). In particular, on the hard surface of the 4% agarose hydrogel and PCL scaffold not coated with hyaluronic acid-catechol, the embryonic salivary gland and its blood vessels (CD31+) did not grow well and showed a high apoptotic rate (Cas3), and low stem cell (c-kit+) cell division activity (Ki-67). In comparison, such apoptosis was not observed on the surface coated with hyaluronic acid-catechol, and high cell division activity and active vascular proliferation of stem cells were observed. In addition, the embryonic salivary glands cultured on the surface coated with hyaluronic acid-catechol were well differentiated into acinar cells expressing Aquaporin 5 and myoepithelial cells expressing alpha-Smooth muscle actin over time to be changed to the same as adult salivary cells. However, on the hard material surface that was not coated with hyaluronic acid-catechol, embryonic salivary glands did not grow properly and did not differentiate well into acinar cells and myoepithelial cells (d).

That is, referring to the results of Comparative Examples and Examples shown in FIG. 7, it can be seen that when a coating layer comprising a hyaluronic acid-catechol compound is provided, excellent culturing characteristics of the embryonic salivary gland are exhibited. In addition, these culture characteristics were excellent in all cases where a coating layer comprising a hyaluronic acid-catechol compound was provided regardless of the type of support.

Accordingly, a tissue regeneration platform can be prepared using various supports by providing a coating layer comprising a hyaluronic acid-catechol compound, and tissue culture such as an embryonic salivary gland can be stably performed therefrom.

While the foregoing has been described with reference to preferred embodiments of the present invention, it will be understood by those skilled in the art or by those of ordinary skill in the relevant art that various changes and modifications can be made therein without departing from the spirit and scope of the invention as set forth in the claims that follow. Accordingly, the technical scope of the present invention should not be limited to the content described in the detailed description of the specification, but should be defined by the claims.

1. A tissue regeneration platform comprising:
a support; and
a coating layer disposed on the support and comprising a hyaluronic acid-catechol compound.

2. The tissue regeneration platform of claim 1, comprising tissue cells for culture provided on the coating layer.

3. The tissue regeneration platform of claim 1, wherein the salivary gland is cultured on the coating layer.

4. The tissue regeneration platform of claim 1, wherein the support is polycarbonate, agarose gel, alginate hydrogel, or polycaprolactone.

5. The tissue regeneration platform of claim 1, wherein the support is a polymer membrane, a polymer gel, or a three-dimensional polymer scaffold.

6. The tissue regeneration platform of claim 1, wherein the hyaluronic acid-catechol compound is represented by the following chemical formula (1): 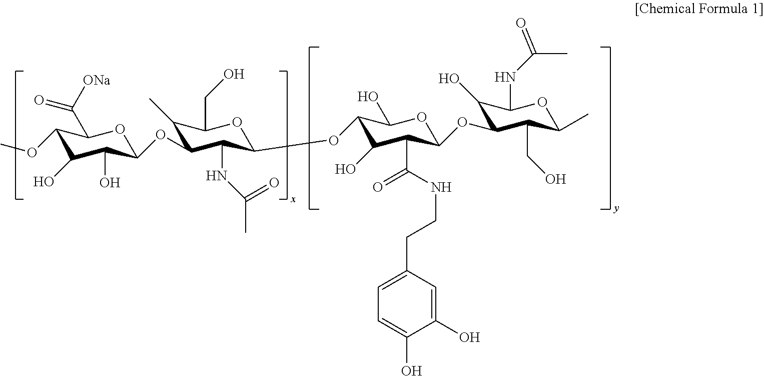
wherein, X and Y are each independently an integer of 1 or more and 300 or less.

7. A method of fabricating a tissue regeneration platform, comprising:
preparing a mixed solution comprising hyaluronic acid-catechol; and
forming a coating layer by providing the mixed solution on a support.

8. The method of claim 7, comprising culturing a salivary gland on the coating layer.

9. The method of claim 7, wherein the support is a polymer membrane, polymer gel, or a three-dimensional polymer scaffold, and
forming the coating layer comprises immersing the support in the mixed solution.
